(19) United States
(12) Patent Application Publication
(10) Pub. No.: US 2007/0156135 A1
(43) Pub. Date: Jul. 5, 2007

(54) System and methods for treating atrial fibrillation using electroporation

(76) Inventors: Boris Rubinsky, Albany, CA (US); Paul Mikus, Coto de Caza, CA (US)

Correspondence Address: HELLER EHRMAN LLP, 275 MIDDLEFIELD ROAD, MENLO PARK, CA, 94025-3506, US

(21) Appl. No.: 11/325,256

(22) Filed: Jan. 3, 2006

(51) Int. Cl. A61B 18/14

(52) U.S. Cl. 606/41

(57) ABSTRACT

The present invention provides an improved system and method for treating atrial fibrillation using electroporation. The system creates transmural lesions in tissue. At least first and second tissue penetrating, mono-polar electrodes are provided that are configured to be introduced at or near an epicardial tissue site of the heart of the patient. A voltage pulse generator is coupled to the first and second mono-polar electrodes. The voltage pulse generator applies sufficient electrical pulses between the first and second mono-polar electrodes to induce electroporation of cells in the epicardial tissue site to create a trausmural lesion, but insufficient to create a thermal damaging effect to a majority of the epicardial tissue site.

### BACKGROUND OF THE INVENTION

1. Field of the Invention

This invention relates generally to systems and methods for treating atrial fibrillation, and more particularly, to systems and methods for treating atrial fibrillation using electroporation.

2. Description of the Related Art

Although atrial fibrillation may occur alone, this arrhythmia often associates with numerous cardiovascular conditions, including congestive heart failure, hypertensive cardiovascular disease, myocardial infarcation, rheumatic heart disease and stroke. Regardless, three separate detrimental sequelae result: (1) a change in the ventricular response, including the onset of an irregular ventricular rhythm and an increase in ventricular rate; (2) detrimental hemodynamic consequences resulting from loss of atroventricular synchrony, decreased ventricular filling time, and possible atrioventricular valve regurgitation; and (3) an increased likelihood of sustaining a thromboembolic event because of loss of effective contraction and atrial stasis of blood in the left atrium.

Atrial arrythmia may be treated using several methods. Pharmacological treatment of atrial fibrillation, for example, is initially the preferred approach, first to maintain normal sinus rhythm, or secondly to decrease the ventricular response rate. While these medications may reduce the risk of thrombus collecting in the atrial appendages if the atrial fibrillation can be converted to sinus rhythm, this form of treatment is not always effective. Patients with continued atrial fibrillation and only ventricular rate control continue to suffer from irregular heartbeats and from the effects of impaired hemodynamics due to the lack of normal sequential atrioventricular contractions, as well as continue to face a significant risk of thromboembolism.

Other forms of treatment include chemical cardioversion to normal sinus rhythm, electrical cardioversion, and RF catheter ablation of selected areas determined by mapping. In the more recent past, other surgical procedures have been developed for atrial fibrillation, including left atrial isolation, transvenous catheter or cryosurgical ablation of His bundle, and the Corridor procedure, which have effectively eliminated irregular ventricular rhythm. However, these procedures have for the most part failed to restore normal cardiac hemodynamics, or alleviate the patient's vulnerability to thromboembolism because the atria are allowed to continue to fibrillate. Accordingly, a more effective surgical treatment was required to cure medically refractory atrial fibrillation of the heart.

On the basis of electrophysiologic mapping of the atria and identification of macroreentrant circuits, a surgical approach was developed which effectively creates an electrical maze in the atrium (i.e., the MAZE procedure) and precludes the ability of the atria to fibrillate. Briefly, in the procedure commonly referred to as the MAZE III procedure, strategic atrial incisions are performed to prevent atrial reentry and allow sinus impulses to activate the entire atrial myocardium, thereby preserving atrial transport function postoperatively. Since atrial fibrillation is characterized by the presence of multiple macroreentrant circuits that are fleeting in nature and can occur anywhere in the atria, it is prudent to interrupt all of the potential pathways for atrial macroreentrant circuits. These circuits, incidentally, have been identified by intraoperative mapping both experimentally and clinically in patients.

Generally, this procedure includes the excision of both atrial appendages, and the electrical isolation of the pulmonary veins. Further, strategically placed atrial incisions not only interrupt the conduction routes of the most common reentrant circuits, but they also direct the sinus impulse from the sinoatrial node to the atrioventricular node along a specified route. In essence, the entire atrial myocardium, with the exception of the atrial appendages and the pulmonary veins, is electrically activated by providing for multiple blind alleys off the main conduction route between the sinoatrial node to the atrioventricular node. Atrial transport function is thus preserved postoperatively, as generally set forth in the series of articles: Cox, Schuessler, Boineau, Canavan, Cain, Lindsay, Stone, Smith, Corr, Chang, and D'Agostino, Jr., The Surgical Treatment of Atrial Fibrillation (pts. 1-4), 101 THORAC CARDIOVASC SURG., 402-426, 569-592 (1991).

While this MAZE III procedure has proven effective in ablating medically refractory atrial fibrillation and associated detrimental sequelae, this operational procedure is traumatic to the patient since substantial incisions are introduced into the interior chambers of the heart. Moreover, using current techniques, many of these procedures require a gross thoracotomy, usually in the form of a median sternotomy, to gain access into the patient's thoracic cavity. A saw or other cutting instrument is used to cut the sternum longitudinally, allowing two opposing halves of the anterior or ventral portion of the rib cage to be spread apart. A large opening into the thoracic cavity is thus created, through which the surgical team may directly visualize and operate upon the heart for the MAZE III procedure. Such a large opening further enables manipulation of surgical instruments and/or removal of excised heart tissue since the surgeon can position his or her hands within the thoracic cavity in close proximity to the exterior of the heart. The patient is then placed on cardiopulmonary bypass to maintain peripheral circulation of oxygenated blood.

Not only is the MAZE III procedure itself traumatic to the patient, but the postoperative pain and extensive recovery time due to the conventional thoracotomy substantially increase trauma and further extend hospital stays. Moreover, such invasive, open-chest procedures significantly increase the risk of complications and the pain associated with sternal incisions. While heart surgery produces beneficial results for many patients, numerous others who might benefit from such surgery are unable or unwilling to undergo the trauma and risks of current techniques.

### SUMMARY OF THE INVENTION

Accordingly, it is an object of the present invention to provide an improved system and method for treating atrial fibrillation.

Another object of the present invention is to provide a system and method for treating atrial fibrillation using electroporation.

These and other objects of the present invention are achieved in a system for creating transmural lesions in tissue. At least first and second tissue penetrating, mono-polar electrodes are provided that are configured to be introduced at or near an epicardial tissue site of the heart of the patient. A voltage pulse generator is coupled to the first and second mono-polar electrodes. The voltage pulse generator applies sufficient electrical pulses between the first and second mono-polar electrodes to induce electroporation of cells in the epicardial tissue site to create a trausmural lesion, but insufficient to create a thermal damaging effect to a majority of the epicardial tissue site.

In another embodiment of the present invention, a system is provided for treating atrial fibrillation. At least first and second mono-polar electrodes are provided that are configured to be introduced at or near a epicardial tissue site of the heart of the patient. A voltage pulse generator is coupled to the first and second mono-polar electrodes. The voltage pulse generator is configured to apply sufficient electrical pulses between the first and second mono-polar electrodes to induce electroporation of cells in the epicardial tissue site to create necrosis of cells of the epicardial tissue site, but insufficient to create a thermal damaging effect to a majority of the epicardial tissue site.

In another embodiment of the present invention, a system is provided for treating atrial fibrillation. A bi-polar electrode is included and configured to be introduced at or near an epicardial tissue site of the heart of the patient. A voltage pulse generator is coupled to the first and second electrodes. The voltage pulse generator is configured to apply sufficient electrical pulses to the bi-polar electrode to induce electroporation of cells in the epicardial tissue site to create necrosis of cells of the epicardial tissue site, but insufficient to create a thermal damaging effect to a majority of the epicardial tissue site.

In another embodiment of the present invention, a system is provided for treating atrial fibrillation. A bi-polar electrode is configured to be introduced at or near a epicardial tissue site of the heart of the patient. A voltage pulse generator is coupled to the bi-polar electrode. The voltage pulse generator is configured to apply sufficient electrical pulses between the bi-polar electrode to induce electroporation of cells in the epicardial tissue site to create necrosis of cells of the epicardial tissue site, but insufficient to create a thermal damaging effect to a majority of the epicardial tissue site.

In another embodiment of the present invention, a system is provided for treating atrial fibrillation. A catheter apparatus includes at least first and second mono-polar electrodes positioned at an inflatable balloon. The balloon is sized to be positioned and expanded at an epicardial tissue site of the heart of a patient. A voltage pulse generator is coupled to the first and second mono-polar electrodes. The voltage pulse generator is configured to apply sufficient electrical pulses between the first and second mono-polar electrodes to induce electroporation of cells in the epicardial tissue site to create necrosis of cells of the epicardial tissue site, but insufficient to create a thermal damaging effect to a majority of the epicardial tissue site.

In another embodiment of the present invention, a system is provided for treating atrial fibrillation. A catheter apparatus includes at least a first bi-polar electrode positioned at an inflatable balloon. The balloon is sized to be positioned and expanded at an epicardial tissue site of the heart of a patient. A voltage pulse generator is coupled to the at least first bi-polar electrode. The voltage pulse generator is configured to apply sufficient electrical pulses to the bi-polar electrode to induce electroporation of cells in the epicardial tissue site to create necrosis of cells of the epicardial tissue site, but insufficient to create a thermal damaging effect to a majority of the epicardial tissue site.

In another embodiment of the present invention, a method is provided for ablating epicardial tissue. An electroporation device is provided with at least first and second mono-polar electrodes. The first and second mono-polar electrodes are positioned at an epicardial tissue site of the heart of a patient. Sufficient electrical pulses are applied to the bi-polar electrode to induce electroporation of cells in the epicardial tissue site to create necrosis of cells of the epicardial tissue site but insufficient to create a thermal damaging effect to a majority of the epicardial tissue site.

### BRIEF DESCRIPTION OF THE DRAWINGS

FIG. 1 is an upper left, posterior perspective view of a human heart incorporating the system and procedure for treatment of medically refractory atrial fibrillation constructed in accordance with the principles of the present invention.

FIG. 2 is a right, antero-lateral perspective view of the human heart incorporating the present invention system and methods thereof.

FIGS. 3A and 3B are schematic diagrams of the atria portion of the heart illustrating the pattern of transmural cryolesions to create a predetermined conduction path in the atrium using the system and methods of the present invention.

FIG. 8 is a perspective view of an embodiment of the present invention that utilizes a catheter and an expandable balloon.

FIG. 9 is a top perspective view of a patient showing use of one embodiment of a system and method of the present invention on the patient.

FIG. 10 is a transverse cross-sectional view of one embodiment of the system of the present invention and the patient, taken through the patient's thorax, showing the relative positioning of the right and left intercostal percutaneous penetrations.

### DETAILED DESCRIPTION

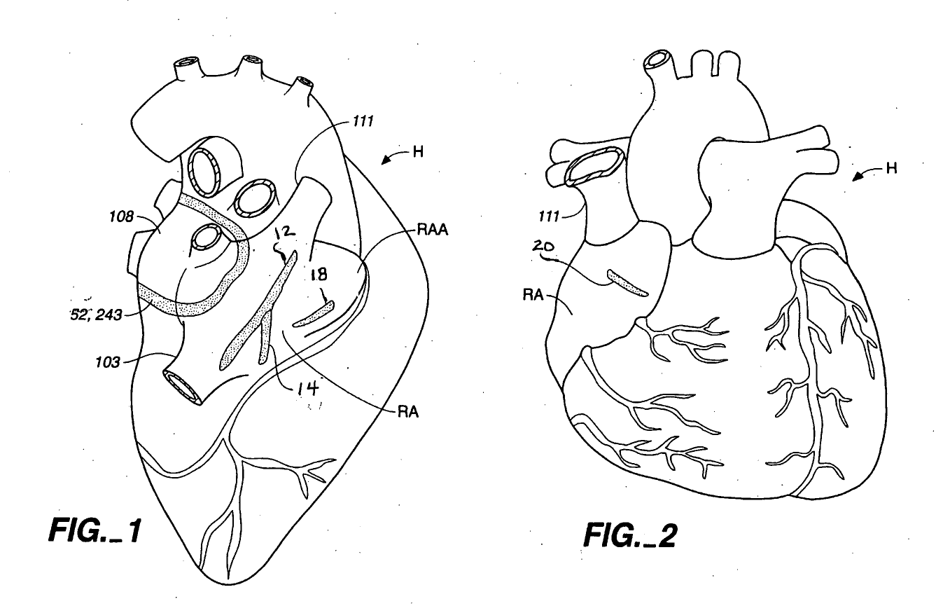

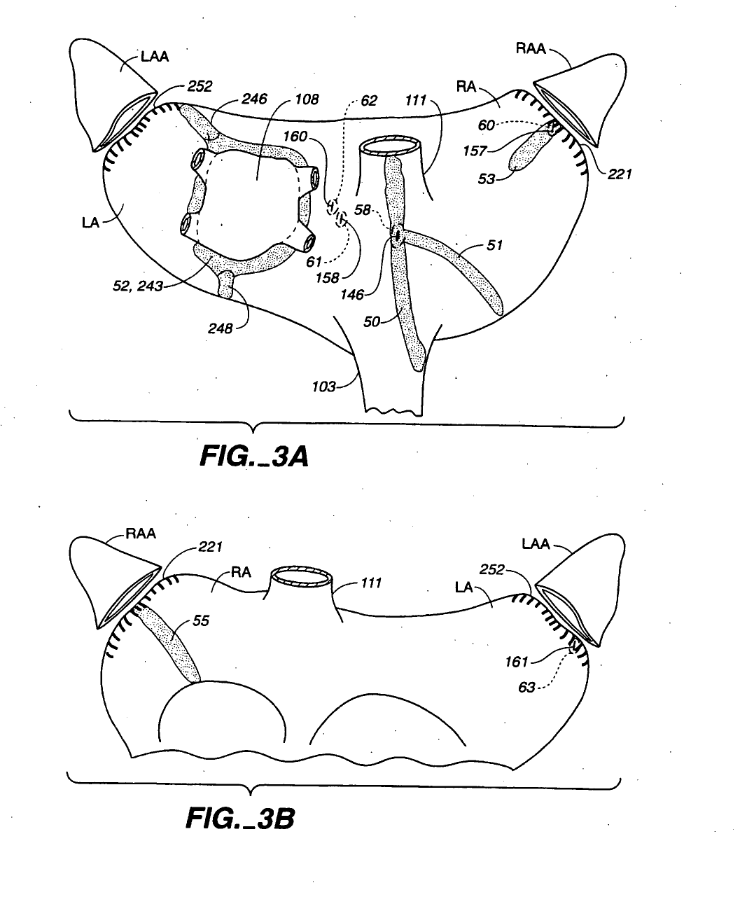

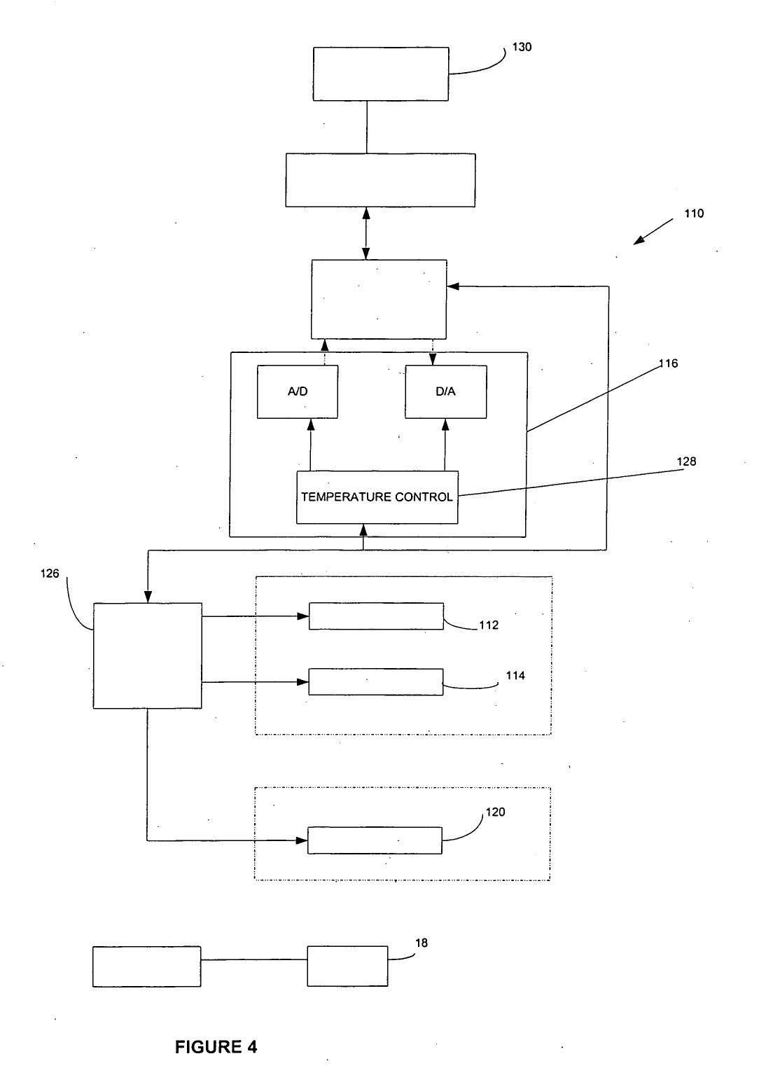

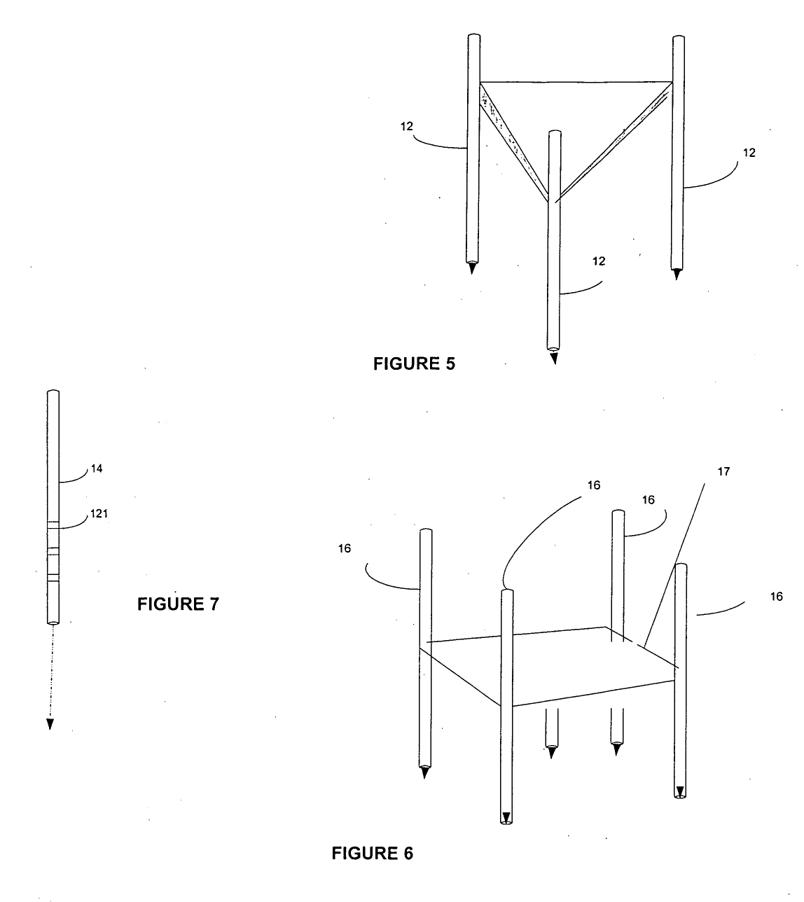

Referring now to FIGS. 1 through 3(b), a human heart H is illustrated. The heart H has a plurality of transmural lesions throughout the right atrium RA and the left atrium LA formed with selected embodiments of the present invention. FIG. 1 illustrates a desired pattern of lesions created on the right atrium RA, including the posterior longitudinal right atrial lesion 12, the tricuspid valve annulus lesion valve annulus lesion 14, the pulmonary vein isolation lesion vein isolation lesion 16 and the perpendicular lesion 18. FIG. 2 illustrates a right, anterior perspective view of the heart H illustrating right atrium RA including a right atrial anteromedial counter lesion 20. The cumulative pattern of lesions reconstruct a main electrical conduction route between the sinoatrial node to the atrioventricular node to postoperatively preserve atrial transport function.

Referring now to FIG. 4, in one embodiment of the present invention a system 110 is provided for creating transmural lesions in a tissue site. At least first and second tissue penetrating or non tissue penetrating, mono-polar electrodes 112 and 114 are provided that are configured to be introduced at or near an epicardial tissue site of the heart H of the patient. A voltage pulse generator 116 is coupled to the first and second mono-polar electrodes 112 and 114. The voltage pulse generator 116 applies sufficient electrical pulses between the first and second mono-polar electrodes 112 and 114 to induce electroporation of cells in the epicardial tissue site to create a trausmural lesion, but insufficient to create a thermal damaging effect to a majority of the epicardial tissue site. It will be appreciated that three or more mono-polar electrodes, as illustrated in FIGS. 5 and 6. In one embodiment, the mono-polar electrodes 112 and 114 are separated by a distance of about 5 mm to 10 cm and they have a circular cross-sectional geometry. One or more additional probes can be provided, including monitoring probes, and the like.

In another embodiment of the present invention, the system 110 includes one or more bi-polar electrodes 120. Each bipolar electrode 120 can have multiple electrode bands 121, illustrated in FIG. 7. The spacing and the thickness of the electrode bands is selected to optimize the shape of the electric field. In one embodiment, the spacing is about 1 mm to 5 cm typically, and the thickness of the electrode bands 20 can be from 0.5 mm to 5 cm.

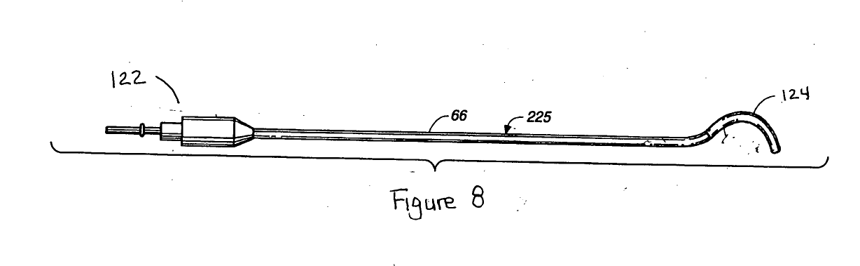

In another embodiment of the present invention, the system 110 is provided for treating atrial fibrillation. In one embodiment, illustrated in FIG. 8, a catheter apparatus 122 can be provided that includes the at least first and second mono-polar electrodes 112 and 114 or the bi-polar electrode 120 which are positioned at an inflatable balloon 124. The balloon 124 is sized to be positioned and expanded at the epicardial tissue site of the heart H of a patient.

The electrodes 112, 114 and 120 are each connected through cables to the voltage pulse generator 116. Returning again to FIG. 4, a switching device 126 can be included. The switching device 126, with software, provides for simultaneous or individual activation of multiple electrodes 112, 114 and 120. The switching device 126 is coupled to the voltage pulse generator 116. In one embodiment, means are provided for individually activating the electrodes 112, 114 and 120 in order to produce electric fields that are produced between pre-selected electrodes 112, 114 and 120 in a selected pattern. The switching of electrical signals between the individual electrodes 112, 114 and 120 can be accomplished by a variety of different means including but not limited to, manually, mechanically, electrically, with a circuit controlled by a programmed digital computer, and the like. In one embodiment, each individual electrode 112, 114 and 120 is individually controlled.

The pulses are applied for a duration and magnitude in order to permanently disrupt the cell membranes of cells at the tissue site. A ratio of electric current through cells at the tissue site to voltage across the cells can be detected, and a magnitude of applied voltage to the tissue site is then adjusted in accordance with changes in the ratio of current to voltage.

In one embodiment, an onset of electroporation of cells at the tissue site is detected by measuring the current. In another embodiment, monitoring the effects of electroporation on cell membranes of cells at the tissue site are monitored. The monitoring can be preformed by image monitoring using ultrasound, CT scan, MRI, CT scan, and the like.

In other embodiments, the monitoring is achieved using a monitoring electrode. In one embodiment, the monitoring electrode is a high impedance needle that can be utilized to prevent preferential current flow to a monitoring needle. The high impedance needle is positioned adjacent to or in the tissue site, at a critical location. This is similar in concept and positioning as that of placing a thermocouple as in a thermal monitoring. Prior to the full electroporation pulse being delivered a “test pulse” is delivered that is some fraction of the proposed full electroporation pulse, which can be, by way of illustration and without limitation, 10%, and the like. This test pulse is preferably in a range that does not cause irreversible electroporation. The monitoring electrode measures the test voltage at the location. The voltage measured is then extrapolated back to what would be seen by the monitoring electrode 18 during the full pulse, e.g., multiplied by 10 in one embodiment, because the relationship is linear). If monitoring for a potential complication at the tissue site, a voltage extrapolation that falls under the known level of irreversible electroporation indicates that the tissue site where monitoring is taking place is safe. If monitoring at that tissue site for adequacy of electroporation, the extrapolation falls above the known level of voltage adequate for irreversible tissue electroporation.

The effects of electroporation on cell membranes of cells at the tissue site can be detected by measuring the current flow.

In various embodiments, the electroporation is performed in a controlled manner, with real time monitoring, to provide for controlled pore formation in cell membranes of cells at the tissue site, to create a tissue effect in the cells at the tissue site while preserving surrounding tissue, with monitoring of electrical impedance, and the like.

The electroporation can be performed in a controlled manner by controlling the intensity and duration of the applied voltage and with or without real time control. Additionally, the electroporation is performed in a manner to provide for modification and control of mass transfer across cell membranes. Performance of the electroporation in the controlled manner can be achieved by selection of a proper selection of voltage magnitude, proper selection of voltage application time, and the like.

Again returning to FIG. 4, the system 110 can include a controller 128 that functions to control temperature of the tissue site. In one embodiment of the present invention, programming of the controller 128 can be in computer languages such as C or BASIC (registered trade mark) if a personnel computer is used for controller 128 or assembly language if a microprocessor is used for the controller 128. A user specified control of temperature can be programmed in the controller 128.

The controller 128 can include a computer, a digital or analog processing apparatus, programmable logic array, a hardwired logic circuit, an application specific integrated circuit (“ASIC”), or other suitable device. In one embodiment, the controller 128 includes a microprocessor accompanied by appropriate RAM and ROM modules, as desired. The controller 128 can be coupled to a user interface 130 for exchanging data with a user. The user can operate the user interface 130 to input a desired pulsing pattern and corresponding temperature profile to be applied to the electrodes 112, 114 and 120.

By way of illustration, the user interface 130 can include an alphanumeric keypad, touch screen, computer mouse, push-buttons and/or toggle switches, or another suitable component to receive input from a human user. The user interface 130 can also include a CRT screen, LED screen, LCD screen, liquid crystal display, printer, display panel, audio speaker, or another suitable component to convey data to a human user. The control board 26 can function to receive controller input and can be driven by the voltage pulse generator 116.

In various embodiment, the voltage pulse generator 116 is configured to provide that each pulse is applied for a duration of about, 5 microseconds to about 62 seconds, 90 to 110 microseconds, 100 microseconds, and the like. A variety of different number of pulses can be applied, including but not limited to, from about 1 to 15 pulses, about eight pulses of about 100 microseconds each in duration, and the like. In one embodiment, the pulses are applied to produce a voltage gradient at the tissue site in a range of from about 50 volt/cm to about 8000 volt/cm.

In various embodiments, the tissue site is monitored and the pulses are adjusted to maintain a temperature of, 100 degrees C. or less at the tissue site, 75 degrees C. or less at the tissue site, 60 degrees C. or less at the tissue site, 50 degrees C. or less at the tissue site, and the like. The temperature is controlled in order to minimize the occurrence of a thermal effect to the tissue site. These temperatures can be controlled by adjusting the current-to-voltage ratio based on temperature.

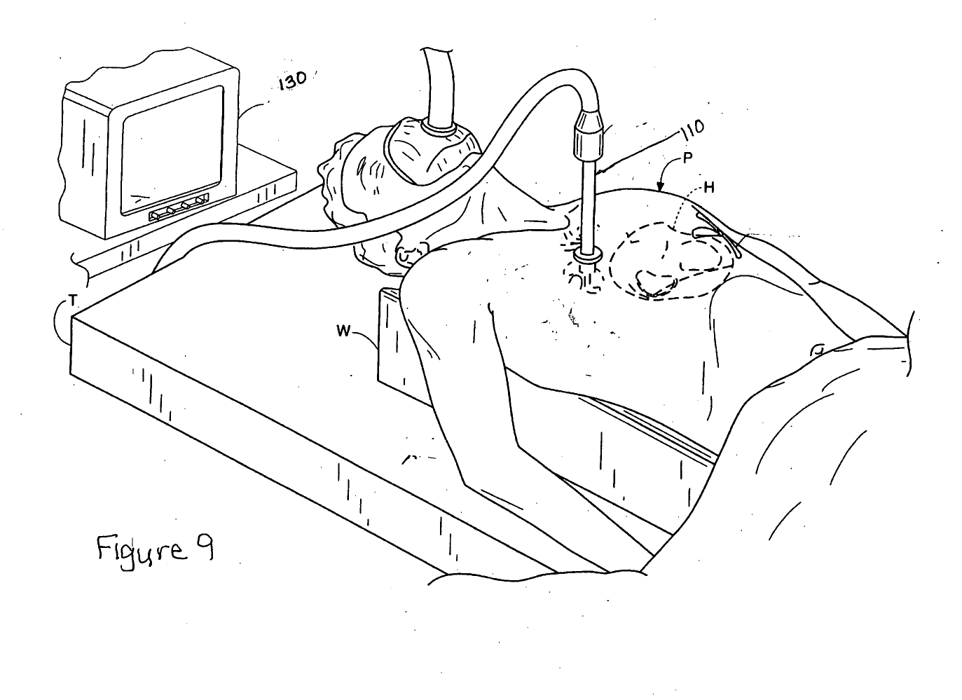

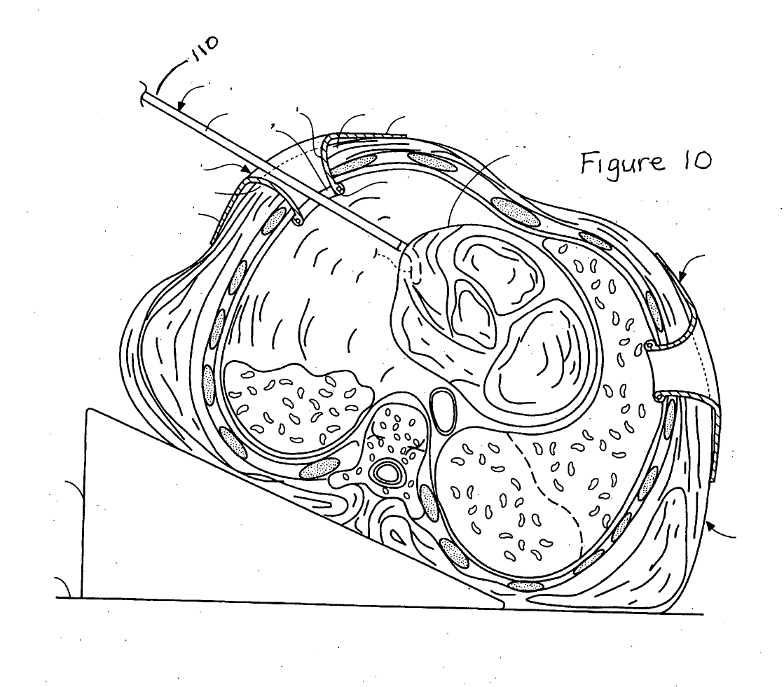

The system 110 can be utilized in both open and closed chest procedures. FIGS. 9 and 10 illustrate the system 110 in a closed-chest, closed-heart surgery positioned in a patient P on an operating table T. The patient is prepared for cardiac surgery in the conventional manner, and general anesthesia is induced. To surgically access the right atrium, the patient is positioned on the patient's left side so that the right lateral side of the chest is disposed upward. A wedge or block W having a top surface angled at approximately 20.degree. to 45.degree. can be used and be positioned under the right side of the patient's body so that the right side of the patient's body is somewhat higher than the left side. It will be understood, however, that a similar wedge or block W can be positioned under the left side of patient P (not shown) when performing the surgical procedure on the left atrium In either position, the patient's right arm A or left arm (not shown) is allowed to rotate downward to rest on table T, exposing either the right lateral side or the left lateral side of the patient's chest.

In one embodiment, a small incision of about 2-3 cm in length is made between the ribs on the right side of the patient P, usually in the third, fourth, or fifth intercostal spaces. When additional maneuvering space is necessary, the intercostal space between the ribs may be widened by spreading of the adjacent ribs. A thoracoscopic access device, including but not limited to a retractor, trocar sleeve, cannula and the like, can provide an access port. The thoracoscopic access device is then positioned in the incision to retract away adjacent tissue and protect it from trauma as instruments are introduced into the chest cavity. Additional thoracoscopic trocars, or the like, can be positioned within intercostal spaces in the right lateral chest inferior and superior to the retractor, as well as in the right anterior (or ventral) portion of the chest if necessary. In other instances, instruments may be introduced directly through small, percutaneous intercostal incisions in the chest.

Once the retractor has been positioned and anchored in the patient's chest, visualization within the thoracic cavity may be accomplished in any of several ways. An endoscope can be positioned through a percutaneous intercostal penetration into the patient's chest, usually through the port of the soft tissue retractor. A video camera can be mounted to the proximal end of the endoscope and is connected to a video monitor for viewing the interior of the thoracic cavity. The endoscope is manipulated to provide a view of the right side of the heart, and particularly, a right side view of the right atrium.

Further, the surgeon may simply view the chest cavity directly through the access port of the retractor. A transesophageal echocardiography can be used, wherein an ultrasonic probe is placed in the patient's esophagus or stomach to ultrasonically image the interior of the heart. A thoracoscopic ultrasonic probe can also be placed through the access device into the chest cavity and adjacent the exterior of the heart for ultrasonically imaging the interior of the heart. An endoscope can also be used that has an optically transparent bulb such as an inflatable balloon or transparent plastic lens over its distal end which is then introduced into the heart. The balloon can be inflated with a transparent inflation fluid such as saline to displace blood away from distal end and may be positioned against a site such a lesion, allowing the location, shape, and size of cryolesion to be visualized.

As a further visualization alternative, an endoscope can be utilized which employs a specialized light filter, so that only those wavelengths of light not absorbed by blood are transmitted into the heart. The endoscope can have a CCD chip designed to receive and react to such light wavelengths and transmit the image received to a video monitor. In this way, the endoscope can be positioned in the heart through the access port and used to see through blood to observe a region of the heart.

In one embodiment, system 110 is used while the heart remains beating. Hence, the trauma and risks associated with cardiopulmonary bypass (CPB) and cardioplegic arrest can be avoided. In other instances, however, arresting the heart may be advantageous. Should it be desirable to place the patient on cardiopulmonary bypass, the patient's right lung is collapsed and the patient's heart is arrested. CPB can be established by introducing a venous cannula into a femoral vein in patient P to withdraw deoxygenated blood therefrom. The venous cannula is connected to a cardiopulmonary bypass system which receives the withdrawn blood, oxygenates the blood, and returns the oxygenated blood to an arterial return cannula positioned in a femoral artery.

A pulmonary venting catheter can also be utilized to withdraw blood from the pulmonary trunk. The pulmonary venting catheter can be introduced from the neck through the interior jugular vein and superior vena cava, or from the groin through the femoral vein and inferior vena cava

For purposes of arresting cardiac function, an aortic occlusion catheter is positioned in a femoral artery by a percutaneous technique such as the Seldinger technique, or through a surgical cut-down. An aortic occlusion catheter is advanced, usually over a guidewire, until an occlusion balloon at its distal end is disposed in the ascending aorta between the coronary ostia and the brachiocephalic artery. Blood can be vented from ascending aorta through a port at the distal end of the aortic occlusion catheter in communication with an inner lumen in the aortic occlusion catheter, through which blood can flow to the proximal end of the catheter. The blood can then be directed to a blood filter/recovery system to remove emboli, and then returned to the patient's arterial system via the CPB system.

When it is desired to arrest cardiac function, the occlusion balloon is inflated until it completely occludes the ascending aorta, blocking blood flow therethrough. A cardioplegic fluid such as potassium chloride (KCI) can be mixed with oxygenated blood from the CPB system and then delivered to the myocardium in one or both of two ways. Cardioplegic fluid can be delivered in an anterograde manner, retrograde manner, or a combination thereof. In the anterograde delivery, the cardioplegic fluid is delivered from a cardioplegia pump through an inner lumen in the aortic occlusion catheter and the port distal to the occlusion balloon into the ascending aorta upstream of the occlusion balloon. In the retrograde delivery, the cardioplegic fluid can be delivered through a retroperfusion catheter positioned in the coronary sinus from a peripheral vein such as an internal jugular vein in the neck.

With cardiopulmonary bypass established, cardiac function arrested, and the right lung collapsed, the patient is prepared for surgical intervention within the heart H. At this point in the procedure, whether cardiac function is arrested and the patient is placed on CPB, or the patient's heart remains beating, the heart treatment procedure and system of the present invention remain substantially similar. The primary difference is that when the procedure of the present invention is performed on an arrested heart, the blood pressure in the internal chambers of the heart is significantly less. It is not necessary to form a hemostatic seal between the device and the heart wall penetration to inhibit blood loss through the penetration thereby reducing or eliminating the need for purse-string sutures around such penetrations.

In order to gain access to the right atrium of the heart, a pericardiotomy is performed using thoracoscopic instruments introduced through the retractor access port. Instruments suitable for use in this procedure, including thoracoscopic angled scissors and thoracoscopic grasping forceps, are described in U.S. Pat. No. 5,501,698, incorporated herein by reference.

After incising a T-shaped opening in the pericardium, about 5.0 cm in length across and about 4.0 cm in length down, the exterior of the heart H is sufficiently exposed to allow the closed-chest, closed-heart procedure to be performed. To further aid in visualization and access to the heart H, the cut pericardial tissue is retracted away from the pericardial opening with stay sutures extending out of the chest cavity. This technique allows the surgeon to raise and lower the cut pericardial wall in a manner which reshapes the pericardial opening and retracting the heart H slightly, if necessary, to provide maximum access for a specific procedure.

Access, suturing, the blockage of blood and the like is further disclosed in U.S. Pat. No. 6,161,543, incorporated herein by reference.

It will be appreciated that the methods and systems 110 of the present invention can be directed to the creation of lesions from the endocardial surfaces of the atria, as well as lesions or portions of the lesions can be created with the endocardial surfaces of the atria. It will be further appreciated that the methods and systems 110 of the present invention can be utilized to treat atrial fibrillation, Wolfe-Parkinson-White (WPW) Syndrome, ventricular fibrillation, congestive heart failure and other procedures in which interventional devices are introduced into the interior of the heart, coronary arteries, or great vessels.

1. A system for creating transmural lesions in tissue, comprising:
at least first and second tissue penetrating, mono-polar electrodes configured to be introduced at or near an epicardial tissue site of the heart of the patient; and
a voltage pulse generator coupled to the first and second mono-polar electrodes and configured to apply sufficient electrical pulses between the first and second mono-polar electrodes to induce electroporation of cells in the epicardial tissue site to create a transmural lesion, but insufficient to create a thermal damaging effect to a majority of the epicardial tissue site.

2. A system for treating atrial fibrilation, comprising:
at least first and second mono-polar electrodes configured to be introduced at or near an epicardial tissue site of the heart of the patient; and
a voltage pulse generator coupled to the first and second mono-polar electrodes and configured to apply sufficient electrical pulses between the first and second mono-polar electrodes to induce electroporation of cells in the epicardial tissue site to create necrosis of cells of the epicardial tissue site but insufficient to create a thermal damaging effect to a majority of the epicardial tissue site.

3. The system of claim 2, further comprising:
a monitoring electrode configured to measure a test voltage delivered to cells in the epicardial tissue site.

4. The system of claim 2, wherein the test voltage is insufficient to create irreversible electroporation.

5. The system of claim 2, further comprising:
at least a third mono-polar electrode, the at least first, second and third mono-polar electrodes forming an array of electrodes.

6. The system of claim 5, wherein the array is configured to be positioned in a surrounding relationship relative to the epicardial tissue site.

7. The system of claim 2, wherein the electroporation is performed in a controlled manner with real time monitoring.

8. The system of claim 2, wherein the electroporation is performed in a controlled manner to provide for controlled pore formation in cell membrane.

9. The system of claim 2, wherein the electroporation is performed in a controlled manner to create a tissue effect in the cells at the epicardial tissue site while preserving surrounding tissue.

10. The system of claim 2, wherein the electroporation is performed in a controlled manner with monitoring of electrical impedance.

11. The system of claim 2, wherein the electroporation is monitored using ultrasound.

12. The system of claim 2, wherein the electroporation is monitored using MRI.

13. The system of claim 2, wherein the electroporation is monitored using a CT scan.

14. The system of claim 2, further comprising:
detecting an onset of electroporation of cells at the epicardial tissue site.

15. The system of claim 14, further comprising:
monitoring the effects of electroporation on cell membranes of cells at the epicardial tissue site.

16. The system of claim 14, further comprising: detecting that the effects of electroporation on cell membranes of cells at the epicardial tissue site continues.

17. The system of claim 2, wherein the electroporation is performed in a controlled manner with controlled intensity and duration of voltage.

18. The system of claim 2, wherein the electroporation is performed in a controlled manner with real time control.

19. The system of claim 2, wherein the electroporation is performed in a manner to provide for modification and control of mass transfer across cell membranes.

20. The system of claim 2, wherein the electroporation is performed in a controlled manner with a proper selection of voltage magnitude.

21. The system of claim 2, wherein the electroporation is performed in a controlled manner with a proper selection of voltage application time.

22. The system of claim 2, wherein the voltage pulse generator is configured to provide that each pulse is applied for a duration of about 5 microseconds to about 62 seconds.

23. The system of claim 2, wherein the voltage pulse generator is configured to provide that each pulse is applied for a duration of about 90 to 110 microseconds.

24. The system of claim 2, wherein the voltage pulse generator is configured to provide that each pulse is applied for a duration of about 100 microseconds.

25. The system of claim 23, wherein the voltage pulse generator is configured to apply from about 1 to 15 pulses.

26. The system of claim 23, wherein the voltage pulse generator is configured to apply about eight pulses of about 100 microseconds each in duration.

27. The system of claim 2, wherein the voltage pulse generator is configured to provide for pulse application to produce a voltage gradient at the epicardial tissue site in a range of from about 50 volt/cm to about 8000 volt/cm.

28. The system of claim 2, wherein a temperature of the epicardial tissue site is monitored and the pulses are adjusted to maintain a temperature of 100 degrees C. or less at the epicardial tissue site.

29. The system of claim 2, wherein a temperature of the epicardial tissue site is monitored and the pulses are adjusted to maintain a temperature of 75 degrees C. or less at the epicardial tissue site.

30. The system of claim 2, wherein a temperature of the epicardial tissue site is monitored and the pulses are adjusted to maintain a temperature of 60 degrees C. or less at the epicardial tissue site.

31. The system of claim 30, wherein the temperature is maintained at 50 degrees C. or less.

32. The system of claim 2, wherein a current-to-voltage ratio is adjusted based on temperature to maintain the epicardial tissue site temperature at 100 degrees C. or less.

33. The system of claim 2, wherein a current-to-voltage ratio is adjusted based on temperature to maintain the epicardial tissue site temperature at 75 degrees C. or less.

34. The system of claim 2, wherein a current-to-voltage ratio is adjusted based on temperature to maintain the epicardial tissue site temperature at 60 degrees C. or less.

35. The system of claim 2, wherein a current-to-voltage ratio is adjusted based on temperature to maintain the epicardial tissue site temperature at 50 degrees C. or less.

36. The system of claim 2, wherein the first electrode is placed at about 5 mm to 10 cm from the second electrode.

37. The system of claim 2, wherein the first and second mono-polar electrodes are circular in shape.

38. The system of claim 2, wherein the voltage pulse generator is configured to provide for pulse application of sufficient duration and magnitude to permanently disrupt cell membranes of cells at the epicardial tissue site.

39. The system of claim 2, wherein a ratio of electric current through cells at the epicardial tissue site to voltage across the cells is detected and a magnitude of applied voltage to the epicardial tissue site is adjusted in accordance with changes in the ratio of current to voltage.

40. A system for treating atrial fibrilation, comprising:
a bi-polar electrode configured to be introduced at or near an epicardial tissue site of the heart of the patient; and
a voltage pulse generator coupled to the first and second electrodes and configured to apply sufficient electrical pulses to the bi-polar electrode to induce electroporation of cells in the epicardial tissue site to create necrosis of cells of the epicardial tissue site, but insufficient to create a thermal damaging effect to a majority of the epicardial tissue site.

41. The system of claim 40, further comprising:
a monitoring electrode configured to measure a test voltage delivered to cells in the epicardial tissue site.

42. The system of claim 40, wherein the test voltage is insufficient to create irreversible electroporation.

43. The system of claim 40, further comprising:
at least a second bipolar electrode

44. The system of claim 40, wherein the electroporation is performed in a controlled manner with real time monitoring.

45. The system of claim 40, wherein the electroporation is performed in a controlled manner to provide for controlled pore formation in cell membrane.

46. The system of claim 40, wherein the electroporation is performed in a controlled manner to create a tissue effect in the cells at the epicardial tissue site while preserving surrounding tissue.

47. The system of claim 40, wherein the electroporation is performed in a controlled manner with monitoring of electrical impedance.

48. The system of claim 40, wherein the electroporation is monitored using ultrasound.

49. The system of claim 40, wherein the electroporation is monitored using MRI.

50. The system of claim 40, wherein the electroporation is monitored using a CT scan.

51. The system of claim 40, further comprising:
detecting an onset of electroporation of cells at the epicardial tissue site.

52. The system of claim 51, further comprising:
monitoring the effects of electroporation on cell membranes of cells at the epicardial tissue site.

53. The system of claim 51, further comprising:
detecting that the effects of electroporation on cell membranes of cells at the epicardial tissue site continues.

54. The system of claim 40, wherein the electroporation is performed in a controlled manner with controlled intensity and duration of voltage.

55. The system of claim 40, wherein the electroporation is performed in a controlled manner with real time control.

56. The system of claim 40, wherein the electroporation is performed in a manner to provide for modification and control of mass transfer across cell membranes.

57. The system of claim 40, wherein the electroporation is performed in a controlled manner with a proper selection of voltage magnitude.

58. The system of claim 40, wherein the electroporation is performed in a controlled manner with a proper selection of voltage application time.

59. The system of claim 40, wherein the voltage pulse generator is configured to provide that each pulse is applied for a duration of about 5 microseconds to about 62 seconds.

60. The system of claim 40, wherein the voltage pulse generator is configured to provide that each pulse is applied for a duration of about 90 to 110 microseconds.

61. The system of claim 40, wherein the voltage pulse generator is configured to provide that each pulse is applied for a duration of about 100 microseconds.

62. The system of claim 60, wherein the voltage pulse generator is configured to apply from about 1 to 15 pulses.

63. The system of claim 60, wherein the voltage pulse generator is configured to apply about eight pulses of about 100 microseconds each in duration.

64. The system of claim 40, wherein the voltage pulse generator is configured to provide for pulse application to produce a voltage gradient at the epicardial tissue site in a range of from about 50 volt/cm to about 8000 volt/cm.

65. The system of claim 40, wherein a temperature of the epicardial tissue site is monitored and the pulses are adjusted to maintain a temperature of 100 degrees C. or less at the epicardial tissue site.

66. The system of claim 40, wherein a temperature of the epicardial tissue site is monitored and the pulses are adjusted to maintain a temperature of 75 degrees C. or less at the epicardial tissue site.

67. The system of claim 40, wherein a temperature of the epicardial tissue site is monitored and the pulses are adjusted to maintain a temperature of 60 degrees C. or less at the epicardial tissue site.

68. The system of claim 67, wherein the temperature is maintained at 50 degrees C. or less.

69. The system of claim 40, wherein a current-to-voltage ratio is adjusted based on temperature to maintain the epicardial tissue site temperature at 100 degrees C. or less.

70. The system of claim 40, wherein a current-to-voltage ratio is adjusted based on temperature to maintain the epicardial tissue site temperature at 75 degrees C. or less.

71. The system of claim 40, wherein a current-to-voltage ratio is adjusted based on temperature to maintain the epicardial tissue site temperature at 60 degrees C. or less.

72. The system of claim 40, wherein a current-to-voltage ratio is adjusted based on temperature to maintain the epicardial tissue site temperature at 50 degrees C. or less.

73. The system of claim 40, wherein the first electrode is placed at about 5 mm to 10 cm from the second electrode.

74. The system of claim 40, wherein the first and second mono-polar electrodes are circular in shape.

75. The system of claim 40, wherein the voltage pulse generator is configured to provide for pulse application of sufficient duration and magnitude to permanently disrupt cell membranes of cells at the epicardial tissue site.

76. The system of claim 40, wherein a ratio of electric current through cells at the epicardial tissue site to voltage across the cells is detected and a magnitude of applied voltage to the epicardial tissue site is adjusted in accordance with changes in the ratio of current to voltage.

77. A system for treating atrial fibrilation, comprising:
a bi-polar electrode configured to be introduced at or near a epicardial tissue site of the heart of the patient; and
a voltage pulse generator coupled to the bi-polar electrode and configured to apply sufficient electrical pulses between the bi-polar electrode to induce electroporation of cells in the epicardial tissue site to create necrosis of cells of the epicardial tissue site but insufficient to create a thermal damaging effect to a majority of the epicardial tissue site.

78. A system for treating atrial fibrilation, comprising:
a catheter apparatus including at least first and second mono-polar electrodes positioned at an inflatable balloon, the balloon being sized to be positioned and expanded at an epicardial tissue site of the heart of a patient;
a voltage pulse generator coupled to the first and second mono-polar electrodes and configured to apply sufficient electrical pulses between the first and second mono-polar electrodes to induce electroporation of cells in the epicardial tissue site to create necrosis of cells of the epicardial tissue site but insufficient to create a thermal damaging effect to a majority of the epicardial tissue site.

79. A system for treating atrial fibrilation, comprising:
a catheter apparatus including at least a first bi-polar electrode positioned at an inflatable balloon, the balloon being sized to be positioned and expanded at an epicardial tissue site of the heart of a patient;
a voltage pulse generator coupled to the at least first bi-polar electrode and configured to apply sufficient electrical pulses to the bi-polar electrode to induce electroporation of cells in the epicardial tissue site to create necrosis of cells of the epicardial tissue site but insufficient to create a thermal damaging effect to a majority of the epicardial tissue site.

80. A method of ablating epicardial tissue, comprising:
providing an electroporation device with at least first and second mono-polar electrodes;
positioning the first and second mono-polar electrodes at an epicardial tissue site of the heart of a patient;
apply sufficient electrical pulses to the bi-polar electrode to induce electroporation of cells in the epicardial tissue site to create necrosis of cells of the epicardial tissue site but insufficient to create a thermal damaging effect to a majority of the epicardial tissue site.

81. A system for treating atrial fibrilation, comprising:
at least first and second mono-polar electrodes configured to be introduced at or near an epicardial tissue site of the heart of the patient, the first electrode having a diameter from about 0.5 mm to about 1 cm and a length of about 2 mm to about 15 cm, the second electrode having a diameter from about 0.5 mm and about 1 cm and a length of about 2 mm and about 15 cm, the first and second electrodes forming linear lesions transmurally; and
a voltage pulse generator coupled to the first and second mono-polar electrodes and configured to apply sufficient electrical pulses between the first and second mono-polar electrodes to induce electroporation of cells in the epicardial tissue site to create necrosis of cells of the epicardial tissue site but insufficient to create a thermal damaging effect to a majority of the epicardial tissue site.

82. The system of 81, wherein at least one of the first and second electrodes has a wire-like geometry.

83. The system of claim 81, wherein at least one of the first and second electrodes is a flat surface electrode or a point electrode.

84. A system for treating atrial fibrilation, comprising:
at least a first bi-polar electrode configured to be introduced at or near an epicardial tissue site of the heart of the patient, the first bi-polar electrode having a diameter from about 0.5 mm to about 1 cm and a length of about 2 mm to about 15 cm; and
a voltage pulse generator coupled to the first bi-polar electrode and configured to apply sufficient electrical pulses to induce electroporation of cells in the epicardial tissue site to create necrosis of cells of the epicardial tissue site but insufficient to create a thermal damaging effect to a majority of the epicardial tissue site.

85. The system of claim 84, further comprising:
at least a second bipolar electrode.

86. A system for treating atrial fibrilation, comprising:
at least a first variable length electrode configured to be introduced at or near an epicardial tissue site of the heart of the patient;
a voltage pulse generator coupled to the first variable length electrode and configured to apply sufficient electrical pulses to induce electroporation of cells in the epicardial tissue site to create necrosis of cells of the epicardial tissue site but insufficient to create a thermal damaging effect to a majority of the epicardial tissue site; and
a sensing device configured to sense that an entire active part of the variable length electrode is on tissue.

87. The system of claim 86, wherein the sensing device is a mechanical sensing device.

88. The system of claim 86, wherein the sensing device is an electrical sensing device.

89. The system of claim 86, wherein the sensing device measure impedance.

90. The system of claim 86, wherein the variable length electrode is a mono-polar electrode.

91. The system of claim 86, wherein the variable length electrode is a bi-polar electrode.
